(19) United States
(12) Patent Application Publication
(10) Pub. No.: US 2022/0079997 A1
(43) Pub. Date: Mar. 17, 2022

(54) METHOD OF TREATING CORNEAL PATHOLOGIES WITH OPHTHALMIC COMPOSITION OF UMBILICAL CORD BLOOD PLASMA

(71) Applicant: Fondazione IRCCS Ca' Granda - Ospedale Maggiore Policlinico, Milano (IT)

(72) Inventors: Paolo Rebulla, Milano (IT); Stefania Villa, Milano (IT); Elisabetta Raspollini, Milano (IT)

(73) Assignee: Fondazione IRCCS Ca' Granda - Ospedale Maggiore Policlinico, Milano (IT)

(21) Appl. No.: 17/533,269

(22) Filed: Nov. 23, 2021

Related U.S. Application Data
(62) Division of application No. 15/517,094, filed on Apr. 5, 2017.

(30) Foreign Application Priority Data
Oct. 6, 2014 (IT) MI2014A001745

(51) Int. Cl. A61K 35/51; A61K 38/18; A61K 47/02; A61K 47/12; A61K 47/26

(52) U.S. Cl. CPC A61K 35/51; A61K 38/1808; A61K 47/26; A61K 47/12; A61K 47/02

(57) ABSTRACT

A method of treating corneal pathologies with an ophthalmic composition of umbilical cord blood plasma, and a method of preparing the ophthalmic composition.

### CROSS-REFERENCE TO RELATED APPLICATIONS

This is a divisional of U.S. application Ser. No. 15/517,094, filed Apr. 5, 2017, which is a 371 National. Stage of PCT/EP2015/073031, filed Oct. 6, 2015, which claims foreign priority to Italian Application No. MI2014A001745, filed Oct. 6, 2014, the entire disclosure of which is incorporated by reference herein.

### BACKGROUND OF THE INVENTION

### 1. Field of the Invention

The present invention concerns a novel ophthalmic preparation for the treatment of corneal pathologies.

### DESCRIPTION OF THE RELATED ART

Preparations for the treatment of corneal pathologies and other disease conditions of the eye are known.

For example, artificial tears are synthetic compositions, whose purpose is to maintain the lubrification of the eye surface. They are in a liquid or gel form, comprising hyaluronic acid, jellifying polymers like carboxymethylcellulose or other similar derivatives and salts. They can also comprise fats and phospholipids in order to mimic the composition of the meibomian gland liquid.

There have also been proposed ophthalmic compositions comprising blood serum; blood serum is defined as the liquid portion of the blood deprived of fibrinogen.

In particular, the use of autologous blood serum has been considered preferable for the higher compatibility and reduced risk of pathogen transmission with respect to allogenic serum. However, it may present some drawbacks due to the presence of altered inflammation mediators, like pro-inflammatory cytokines, and autoantibodies, which can be present, thereby exposing the patient to possible harmful agents.

In addition, under certain circumstances the collection of blood sample could be problematic, like from elderly or child patients.

More recently, there has also been proposed the use of umbilical cord blood serum for the preparation of eye drops. The prior-art publication of Kyung-Chul Yoon et al. (Cornea, 2006) describes the effective application of umbilical cord blood serum eye drops for the treatment of 31 patients affected by severe dry eye syndrome.

Han-Jin Oh et al. (Current Eye Research, 2012) discloses experiments performed with umbilical cord blood serum eye drops in a mouse model of ocular chemical burn.

Versura et al. (Cornea, April 2013) disclose quality controls and procedures for preparing standardized eye drops from umbilical cord blood serum to be used in patients affected by corneal pathologies.

The purpose of the present invention is to provide an alternative source of biological material for the preparation of an ophthalmic composition for treating pathological conditions of the eye.

### SUMMARY OF THE INVENTION

It has been surprisingly found that plasma from umbilical cord blood can be used as a source for the preparation of ophthalmic compositions for treating corneal pathologies.

### OBJECT OF THE INVENTION

According to a first object, the present invention discloses the use of plasma from umbilical cord blood as a medicament for the treatment of corneal pathologies.

In particular, the plasma is used for medical or veterinary applications.

In a further object, ophthalmic compositions comprising umbilical cord blood plasma are disclosed.

It is an additional object of the present invention to provide a method for obtaining a preparation comprising umbilical cord blood plasma.

### DETAILED DESCRIPTION OF THE INVENTION

In accordance with a first object, it is disclosed the use of plasma from umbilical cord blood as a medicament for the treatment of corneal pathologies.

In particular, the medicament can be applied to mammals and preferably to humans.

In an alternative embodiment of the invention, the medicament can also be used for the treatment of non-human mammals, preferably selected in the group comprising dogs, cats and horses.

According to the present description, plasma is defined as the liquid portion of blood.

For the purposes of the present invention, corneal pathologies are meant to comprise, for example: the dry eye syndrome, the graft-versus-host disease (GVHD), lesions caused by chemical burns, neurotrophic keratitis, Sjogren's syndrome, systemic sclerosis, rheumatoid arthritis and autoimmunity.

In a preferred embodiment, the dry eye syndrome is accompanied by one or more of other disease conditions like: lacrimal fluid reduction, tear deficiency, xerosis of the eye, kerato-conjunctivitis sicca, Stevens-Johnson syndrome, pemphigoid of the eye, marginal blepharitis, allergic conjunctivitis, ulcerations or may be the consequence of viral conjunctivitis, cornea surgery including laser in situ keratomileusis (LASIK), cataract surgery, contact lens wearing, video display terminal working activities or maybe age-related.

The source of the presently disclosed plasma is represented by the blood remaining in the placenta after birth.

In case the invention is applied to human, it is represented by the infant blood in the placenta after childbirth.

In order to be used for the treatment of corneal pathologies, it shall be processed according to a further object of the invention.

In particular, an isolated sample of umbilical cord blood is first collected and contacted with an anticoagulant agent or a mixture of anticoagulants.

According to a preferred embodiment, said anticoagulant agent is selected in the group comprising: citrate, phosphate, dextrose.

According to a further preferred embodiment, the mixture comprises citrate, phosphate and dextrose (known as CPD solution).

More particularly, the CPD solution may have the following composition:

| | Quantity |
|---|---|
| | (g per 100 ml of |
| Component | anticoagulant mixture) |
| Sodium citrate di-hydrate | 2.63 |
| Sodium citrate hydrate | 0.327 |
| Monosodium di-hydrate phosphate | 0.251 |
| Dextrose monohydrate | 2.55 |
| Water for injection | q.b. to 100 ml |

The anticoagulant agent or mixture of anticoagulant agents and the amounts thereof are comprised between about 10-60% (volume/volume) of composition.

In a preferred embodiment, said agent or mixture of agents are comprised in an amount of about 15 or 20 or 25 or 30 or 35 or 40 or 45 or 50 or 55% (volume/volume) and even more preferably of about 50% (volume/volume).

The composition is then subjected to centrifugation.

According to a first embodiment of the invention, the centrifugation is performed at a rotation of between about 1,500 to 2,500 g, preferably of between about 1,700 to 2,300 g and even more preferably of between about 1,900 to 2,100 g. In a preferred aspect, said step is performed for a period of between about 10 to 20 minutes, preferably of between about 13 to 17 minutes and even more preferably of between about 14 to 16 minutes.

Then the supernatant plasma is transferred into an empty bag (having suitable properties for storing and containing such product), from which the ophthalmic compositions of the invention are prepared.

For example, for a suitable formulation, the umbilical cord blood plasma preparation is preferably diluted to a concentration of epidermal growth factor (EGF) of about 0.10-0.20 ng/ml.

In a preferred embodiment, the dilution is performed in order to obtain a final concentration of EGF of about 0.15 ng/ml. The above reported final concentrations take into consideration the dilution occurred to the collected sample in view of the addition of the anti-coagulant preparation.

Said dilution is preferably between 1:2 and 1:1,3.

According to an alternative embodiment, the preparation of the plasma sample is a dual-step procedure, comprising, before the above described step, a preliminary centrifugation.

In particular, said preliminary step is performed at a rotational speed of between about 100-400 g, preferably of between about 120-350 g and more preferably of between about 150-250 g.

In a preferred embodiment, the centrifugation step is performed for a period of between about 5-20 minutes, preferably of between about 7-15 minutes and even more preferably of between about 9-11 minutes.

As an advantage, the alternative dual-step procedure allows to obtain not only the umbilical cord blood plasma, but also a platelet concentrate suitable for the preparation of cord blood platelet gel.

The preparation obtained according to the methods disclosed can be administered for the treatment of corneal pathologies, like, for instance, the dry eye syndrome, the graft-versus-host disease (GVHD), lesions caused by chemical burns, neurotrophic keratitis, Sjogren's syndrome, systemic sclerosis, rheumatoid arthritis and autoimmunity.

In particular, it can be administered to humans and non-human mammals, like, for instance, cats, dogs and horses.

Therefore, the present invention may find application also in the veterinary field.

According to a further embodiment, the invention provides compositions for the treatment of corneal pathologies.

In particular, said compositions may be in the form of eye drops, ointment, spray or other suitable formulations.

In a preferred embodiment, the ophthalmic compositions are designed as multiple aliquots each of which is suitable for a single-dose self-administration.

For said purpose, aliquots of about 2 ml of plasma preparation, especially for eye drops, are preferably prepared.

The protocol for preparing the ophthalmic composition of the invention comprises suitable upstream procedures before the centrifugation or the preliminary centrifugation.

In fact, donors need to be selected and tests are to be performed on the samples for checking the absence of pathologies and/or the presence of specific markers.

For example, tests for the identification of markers of syphilis, HIV, HCV, HBV, bacteria and fungi are performed. According to a preferred embodiment of the invention, in fact, the sample of umbilical cord blood, which is used as the source for the preparation of the ophthalmic compositions of the invention, is the one which is not suitable for hematopoietic transplantation.

In particular, after collecting the umbilical cord blood sample and excluding the presence of standard criteria that would exclude the sample from clinical use, the amount of biological components is checked and if the count of total nucleated cells (a proxy of hematopoietic stem cell count) is insufficient for hematopoietic transplantation purposes, then the sample is processed according to the above description.

In particular, the number of hematopoietic stem cells is considered insufficient for transplantation purposes if the count of total nucleated cells before processing is below 1000-1500×10⁶.

In any event, it is preferred that the umbilical cord blood sample processed according to the present invention is of at least 40-50 ml.

According to a further embodiment of the invention, it is disclosed the use of umbilical cord blood plasma in the treatment of corneal pathologies in non-human mammals. For said purpose, cord blood is from non-human mammals, which in a preferred embodiment are selected in the group comprising dog, cat and horse.

For the preparation of said compositions, the same method above disclosed can be applied.

### Example

### Preparation of an Eye Formulation from Umbilical Cord Blood Plasma

A sample of human umbilical cord blood has been collected from a suitable donor into a bag containing the anticoagulant agent:

| | Quantity |
|---|---|
| Component | (g per 100 ml) |
| Sodium citrate di-hydrate | 2.63 |
| Sodium citrate hydrate | 0.327 |
| Monosodium di-hydrate phosphate | 0.251 |
| Dextrose monohydrate | 2.55 |
| Water for injection | q.b. to 100 ml |

After checking that the presence of biological components is insufficient for hematopoietic transplantation, the sample has been subjected to a first centrifugation at low speed (220 g for 10 minutes).

The red blood cells have then been separated and the supernatant platelet-rich plasma has been subjected to a centrifugation step at high speed (2,000 g for 15 minutes). The platelet-rich fraction has been separated and the concentration of EGF checked in the platelet-poor plasma fraction.

Dilution of the sample has then been performed to a final EGF concentration of 0.15 ng/ml.

Aliquots of 2 ml each have been prepared for use.

From the above description, the advantages of the proposed invention will be immediately evident.

In particular, umbilical cord blood plasma is a surprisingly superior biological source of growth factors, which contributes to an unexpected increase in the rate of healing.

In addition to that, collection of peripheral autologous blood from patients can thus be avoided.

Accordingly, many problems connected to the poor compliance from certain categories of patients is advantageously overcome.

As a further advantage of the present invention, the preparation of eye drops or other ophthalmic compositions from umbilical cord blood plasma is fully and better integrated within the daily procedures in hospitals.

In fact, the collected cord blood from donation can serve only to a limited extent for transplantation purposes, because in only 10% of the cases the amount of haemopoietic stem cells in the sample renders it suitable for the treatment of blood diseases.

It is therefore an important advantage of the presently disclosed invention, the possibility of using a product which would be otherwise discarded in 90% of the cases.

As a further important aspect, the present invention does not require modification of the existing protocols for a separate collection of samples, that is on the other side required for collecting and handling serum samples.

In particular, there can be applied conditions comprising the use of known bags, which comprise anti-coagulant agents.

In addition, the method for obtaining the disclosed plasma sample can at the same time allow the preparation of cord blood platelet gel, which is a product that can be used for other useful purposes.

As above disclosed, the invention can find application for the treatment of pathologies both in the medical and in the veterinary field.

1. A method for the treatment of corneal pathologies comprising administration of an ophthalmic composition comprising a platelet-poor fraction of umbilical cord blood plasma derived from blood remaining in a placenta after birth.

2. The method for the treatment of corneal pathologies according to claim 1, wherein the platelet-poor fraction of umbilical cord blood plasma has a concentration of between about 0.20-0.40 ng/ml of epidermal growth factor (EGF).

3. The method for the treatment of corneal pathologies according to claim 1, wherein the platelet-poor fraction of umbilical cord blood plasma has a concentration of about 0.30 ng/ml of epidermal growth factor (EGF).

4. The method for the treatment of corneal pathologies according to claim 1 for the treatment of humans or non-human mammals.

5. The method for the treatment of corneal pathologies according to claim 4, wherein said non-human mammals are selected from the group consisting of: cat, dog and horse.

6. The method for the treatment of corneal pathologies according to claim 5, wherein said corneal pathologies comprise any of: dry eye syndrome, the graft-versus-host disease (GVHD), lesions caused by chemical burns, neurotrophic keratitis, Sjogren's syndrome, systemic sclerosis, rheumatoid arthritis, autoimmunity.

7. The method for the treatment of corneal pathologies according to claim 6, wherein the ophthalmic composition comprises a platelet-poor fraction of umbilical cord blood plasma derived from blood remaining in a placenta after birth and an anticoagulant agent, the composition having a concentration of epidermal growth factor (EGF) of about 0.10-0.20 ng/ml.

8. The method for the treatment of corneal pathologies according to claim 7, wherein said anticoagulant agent is selected from the group comprising citrate, phosphate, dextrose or a mixture thereof.

9. The method for the treatment of corneal pathologies according to claim 7, wherein said anticoagulant agent has the following composition:

| | Quantity |
|---|---|
| | (g per 100 ml of |
| Component | anticoagulant mixture) |
| Sodium citrate di-hydrate | 2.63 |
| Sodium citrate hydrate | 0.327 |
| Monosodium di-hydrate phosphate | 0.251 |
| Dextrose monohydrate | 2.55 |
| Water for injection | up to 100 ml |

10. The method for the treatment of corneal pathologies according to claim 8, wherein said anticoagulant agent is comprised in an amount of between about 50% (volume/volume).

11. The method for the treatment of corneal pathologies according to claim 8, wherein said ophthalmic composition has a concentration of about 0.15 ng/ml of EGF.

12. A method for preparing the ophthalmic composition according to claim 1, comprising the step of contacting an isolated sample of umbilical cord blood derived from blood remaining in the placenta after birth with an anticoagulant agent or a mixture of anticoagulant agents, and the step of subjecting the obtained preparation to centrifugation.

13. The method for preparing the ophthalmic composition according to claim 12, wherein the centrifugation is performed at a rotational speed of between about 1500-2500 g, for a period of between about 10-20 minutes.

14. The method for preparing the ophthalmic composition according to claim 12, wherein the centrifugation is performed at a rotational speed of between about 1700-2300 g, for a period of between about 13-17 minutes.

15. The method for preparing the ophthalmic composition according to claim 12, wherein the centrifugation is performed at a rotational speed of between about 1900-2100 g, for a period of between about 14-16 minutes.

16. The method for preparing the ophthalmic composition according to claim 12, which is preceded by a preliminary step of centrifugation at a rotational speed of between about 100-400 g, for a period of between about 5-20 minutes.

17. The method for preparing the ophthalmic composition according to claim 12, which is preceded by a preliminary step of centrifugation at a rotational speed of between about 150-350 g, for a period of between about 7-15 minutes.

18. The method for preparing the ophthalmic composition according to claim 12, which is preceded by a preliminary step of centrifugation at a rotational speed of between about 150-250 g, for a period of between about 9-11 minutes.

19. The method for preparing the ophthalmic composition according to claim 12, further comprising the step of diluting the final preparation to a concentration of about 0.10-0.20 ng/ml of epidermal growth factor (EGF).

20. The method for preparing the ophthalmic composition according to claim 16, comprising, before the centrifugation step or the preliminary centrifugation step, a step for the selection of the isolated sample of umbilical cord blood, which includes checking the total nucleated cell count as a proxy of the content of the haemopoietic stem cells count suitable for transplantation and optionally the testing for markers for syphilis, HIV, HCV, HBV, bacteria, fungi.
